(19) United States
(12) Patent Application Publication
(10) Pub. No.: US 2007/0134801 A1
(43) Pub. Date: Jun. 14, 2007

(54) Method and apparatus for analysing a liquid

(76) Inventors: Jonathan Andrew Fuller, Perthshire (GB); Nasr-Eddine Djennati, Altrincham (GB); Andrew Mitchell, Lancashire (GB)

Correspondence Address: KAPLAN GILMAN GIBSON & DERNIER L.L.P., 900 ROUTE 9 NORTH, WOODBRIDGE, NJ, 07095, US

(21) Appl. No.: 10/578,757

(22) PCT Filed: Nov. 5, 2004
(86) PCT No.: PCT/GB04/04676

§ 371 Date: Oct. 26, 2006

(30) Foreign Application Priority Data
Nov. 7, 2003 (GB) 0326027.0

(51) Int. Cl. G01N 33/86

(52) U.S. Cl. 436/69

(57) ABSTRACT

An apparatus and method for determining the coagulation status of a liquid, especially blood. The apparatus includes a chamber (1) for holding a quantity of said liquid, a body (2) disposed in the chamber and a magnetic device (4,5), the magnetic device co-operating with said chamber and being arranged in use to provide a magnetic field which causes the body to migrate to and from within the chamber through uncoagulated liquid. The body is other than a particle, and the cross-sectional area of the body measured in a pane generally perpendicular to its normal direction of travel in use may be at least half that of the chamber in the same plane.

The present invention relates to a method and apparatus for determining the coagulation status of a liquid and to the use of at least one magnetic field sensor to detect the movement and/or position of a body within a liquid in order to determine the coagulation status of a liquid.

More particularly but not exclusively there is disclosed a method and apparatus for analysing a biological fluid sample to determine a disturbance of haemostasis resulting in a change of viscosity. In embodiments, the method and apparatus may be used to determine the coagulation or prothrombin time (PT) of a sample of blood or plasma. This may be expressed as an Intemationalised Normalised Ratio (INR). Other disturbances of haemostasis that may be determined include measurement of the degree of platelet aggregation, the rate or amount of clot formation and/or clot dissolution, the time required for forming a fibrin clot, the activated partial thromboplastin time (APTT), the activated clotting time (ACT), the protein C activation time (PCAT), the Russell's viper venom time (RVVT) and the thrombin time (TT).

Coagulation of blood in a living body, thrombosis, is one of the leading causes of death world-wide. People who suffer from cardiac or vascular diseases and patients that have undergone surgical procedures are at risk of developing blood clots that may result in life-threatening clinical conditions. Such people are often treated with blood-thinning or anticoagulant drugs such as warfarin or aspirin. However, the amount of anticoagulant in the bloodstream must be maintained at the proper level: too little may result in unwanted clotting whilst too much can result in haemorrhaging with life threatening consequences. As a result routine coagulation screening tests have been developed in order to evaluate the coagulation status of blood or plasma.

Various apparatus has developed for use in the laboratory and as point of care testing (POCT). In addition to this, devices have been developed which allow patients to home-monitor their blood coagulation, such as the CoaguChek Plus™ coagulation meter.

It is an object of embodiments of the present invention to provide an alternative apparatus and method for monitoring blood coagulation.

According to a first aspect of the invention there is provided apparatus for determining the coagulation status of a liquid, the apparatus comprising a chamber for holding a quantity of said liquid, a body disposed in the chamber and a magnetic device, the magnetic device co-operating with said chamber and being arranged in use to provide a magnetic field which causes the body to migrate to and fro within the chamber through uncoagulated liquid, wherein the body is other than a particle.

According to a second aspect of the invention there is provided apparatus for determining the coagulation status of a liquid, the apparatus comprising a chamber for holding a quantity of said liquid, a body disposed in the chamber and a magnetic device, the magnetic device co-operating with said chamber and being arranged in use to provide a magnetic field which causes the body to move to and fro within the chamber through uncoagulated liquid, wherein the cross-sectional area of the body measured in a plane generally perpendicular to its normal direction of travel in use is at least half that of the chamber in the same plane.

According to a third aspect of the present invention there is provided a method of determining the coagulation status of a liquid sample comprising the steps of: providing a sample of liquid in a chamber containing a body and applying a magnetic field to the chamber to cause the body to move to and fro within the chamber through uncoagulated liquid, wherein the body is other than a particle.

According to a fourth aspect of the present invention there is provided a method of determining the coagulation status of a liquid sample comprising the steps of: providing a sample of liquid in a chamber containing a body; applying a magnetic field to the chamber to cause the body to move to and fro within the chamber through uncoagulated liquid, wherein the cross-sectional area of the body measured in a plane generally perpendicular to its normal direction of travel in use is at least half that of the chamber in the same plane.

According to a fifth aspect of the present invention there is provided the use of at least one magnetic field sensor to detect the movement and/or position of a body within a liquid disposed in a chamber in order to determine the coagulation status of a liquid, the body comprising a material which experiences a force when placed in a magnetic field, wherein the body is other than a particle.

The method may comprise cyclically providing a first and a second magnetic field, said first magnetic causing the body to move in a first direction and said second magnetic field causing the body to move in a second direction, the second direction being opposite to the first. The first and second magnetic fields may be provided from different spatial locations, or from the same spatial location. Each field is preferably provided as a short pulse, with a field free period between the short pulses. The duration of each pulse may be less than 500 ms, and in one embodiment is between 10 and 250 ms. The body may be caused to move to and fro within the chamber at a frequency of between 0.1 and 10 Hz.

This magnitude of the magnetic field is preferably less than 25 mT, more preferably less than 15 mT, and still more preferably less than 10 mT.

Means may be provided to detect movement and/or position of the body within the chamber. Such means preferably comprises a magnetic field sensor such as a Hall Effect sensor, magnetorestrictive sensor, search coil or any other means of detecting a change in magnetic field. In an embodiment two or more sensors are provided, each one associated with a respective chamber. In operation the magnetic field measured by the sensor will, amongst other things, be affected by the position of the body relative to the sensor. Thus, the output of a sensor can be used to determine position and/or movement of the body in the chamber. The sensor may also respond to the rate of change of magnetic field detecting motion, not position

The chamber may be of any suitable volume. In an embodiment the free volume within the chamber when the chamber contains the body is less than 10 μl, in another embodiment it is less than 5 μl. The chamber may be of any convenient shape. In an embodiment the chamber is formed in a disposable support strip which is removable from the apparatus. Fluid may be introduced into the chamber by any convenient means, including capillarity. The chamber may be of any suitable material that enables the test to be performed and may be constructed of a non-magnetic material.

In an embodiment a filling device for filling the container includes a capillary. In another, the filling device includes a plunger. The apparatus may comprise more than one chamber. The chamber may be divided into two, three or more compartments.

The chamber may be elongate. In an embodiment it is between 3 and 5 mm in length. The chamber may have any suitable cross-section, for example substantially circular, rectangular or square. The chamber is preferably of substantially uniform cross-section.

The body may be elongate. The body may have a cross-section of substantially the same shape as the cross-section of the chamber. In this case the body is preferably dimensioned in cross-section so that there is a clearance of at least 50 microns between the body and walls of the chamber. The clearance may be less than 300 microns. The cross-sectional area of the body, taken transversely to the intended direction of travel of the body within the chamber in use may be at least half that of the corresponding cross-section of the chamber. The length of the chamber and body may be chosen so that the body can move at least 0.5 mm to and fro within the chamber. In one embodiment the body can move a maximum of 2 mm to and fro within the chamber. The body may have a close sliding fit within the chamber.

The body preferably comprises a material which experiences a force when placed in a magnetic field and may be ferromagnetic. In another it is paramagnetic. In yet another it is superparamagnetic. Where the body is ferromagnetic is may comprise a rare earth magnet. Where the body is ferromagnetic a lower external field may be applied to move the body within the chamber, than for paramagnetic and superparamagnetic bodies.

In an embodiment the chamber contains only a single body. In another more the chamber contains more than one body.

A clotting reagent may be disposed in the chamber prior to introduction of a sample to be analysed. Suitable reagents for measurement of PT include, Thromborel S™ and Innovin™ (produced by Dade) and ThromboTest™ (produced by Axis Shield).

Where more than one chamber or compartment is employed, the reagents disposed in each may be different such as to alter the clotting rate and/or times. Alternatively, one of the compartments or chambers may have no reagent present such that the clotting time independent of clotting reagent may additionally be measured.

As a further alternative, one of the compartments may have a reagent present which inhibits the clotting of the sample such that it does not clot within the time frame of the test.

The magnetic device may comprise a single electromagnet. Alternatively it may comprise two spaced apart electromagnets. The electromagnets may be disposed on mutually opposite sides of the chamber. Alternatively they may be disposed on the same side of the chamber. Each electromagnet may comprise a solenoid or coil. The solenoids or coils may be substantially coaxial.

In one described embodiment the electromagnets are activated alternately with a direct current, to produce a constant field. The magnitude of field produced by one magnet may be greater than the other.

The apparatus may include circuitry for measuring the time elapsed from introduction of a sample until coagulation is detected. The apparatus may comprise a control means, which may comprise a microprocessor. The apparatus may comprise a display, operative to display information to a user. The apparatus may display a clotting time and/or an INR value.

The apparatus may comprise means for heating the chamber, to maintain a sample being analysed at a desired temperature, for example 37° C.

In order that the invention may be more clearly understood embodiments thereof will now be described by way of example with reference to the accompanying drawings of which:

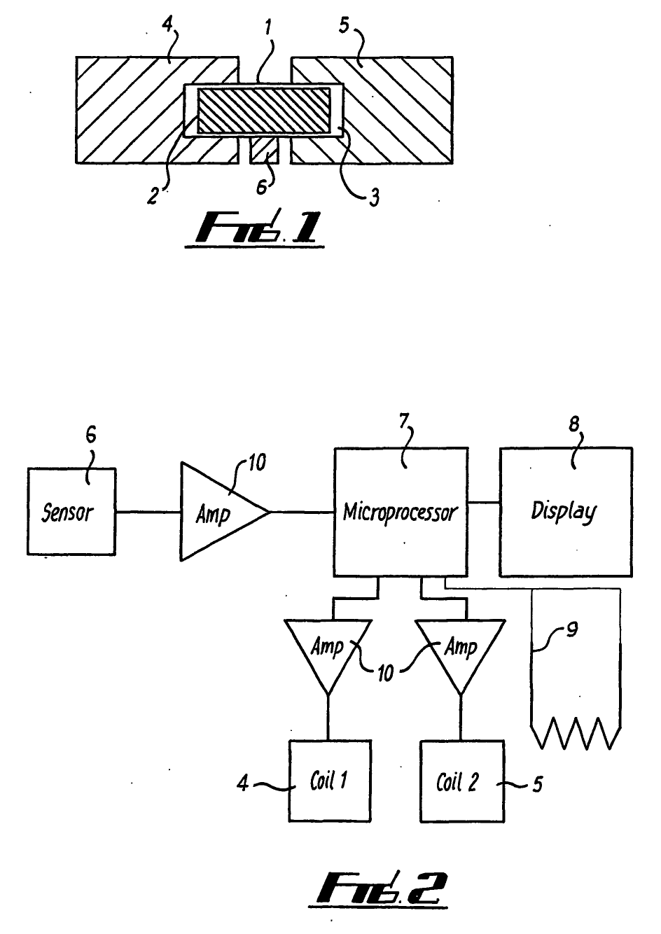

FIG. 1 is a schematic diagram of apparatus embodying the invention;

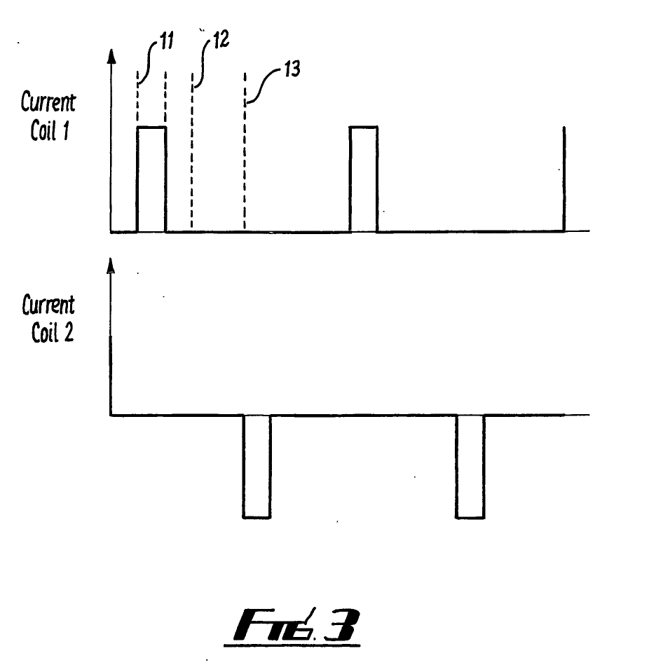

FIG. 2 is a block circuit diagram of the apparatus of FIG. 1;

FIG. 3 shows current against time for the solenoids of the apparatus of FIG. 1 during operation;

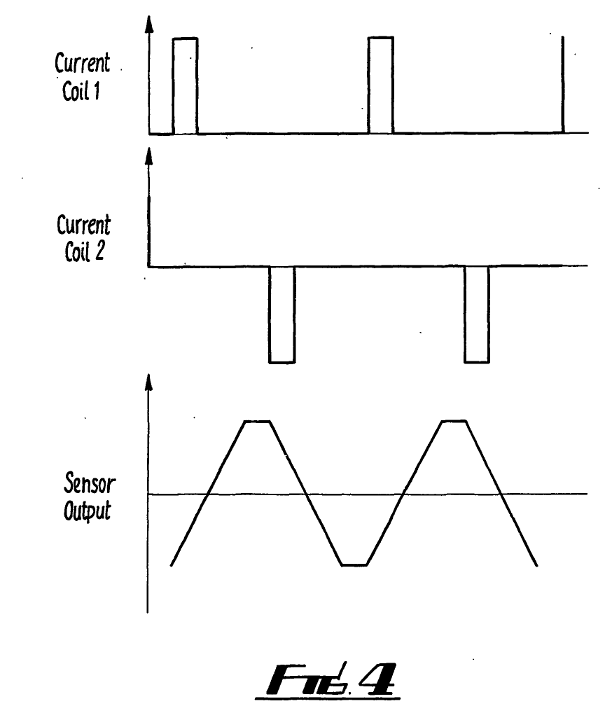

FIG. 4 shows the current against time of FIG. 3 together with sensor output for an unclotted sample;

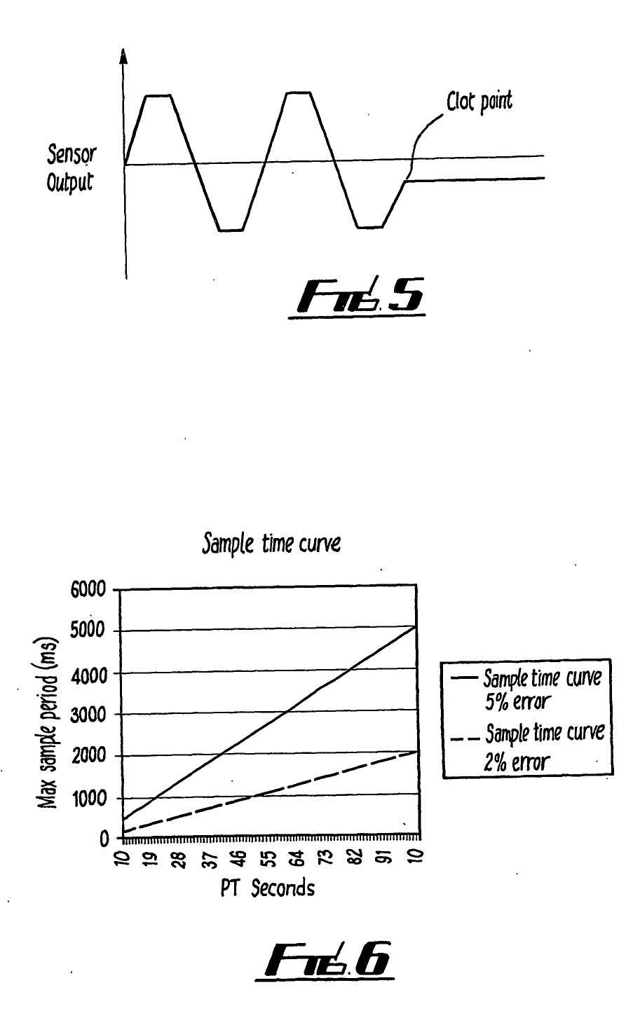

FIG. 5 shows how the sensor output of FIG. 4 changes on clotting of a sample;

FIG. 6 is a graph of sample period against measured prothrombin time for measurements taken with different errors;

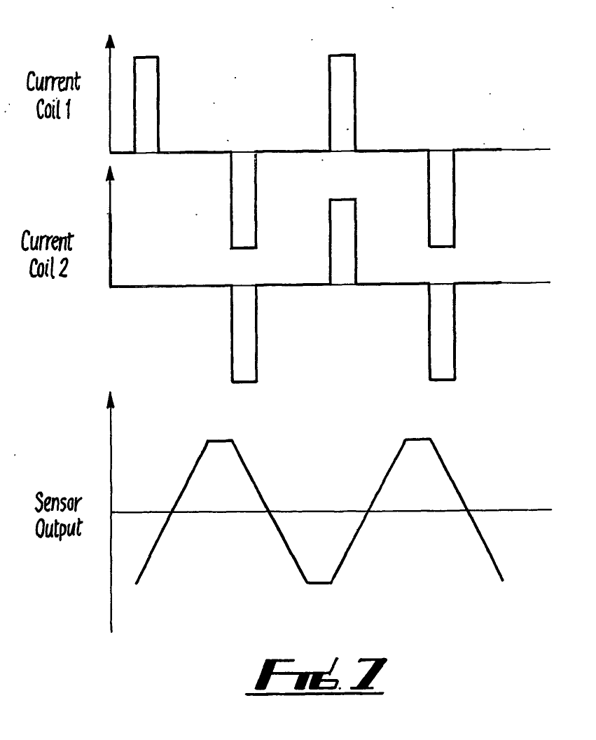

FIG. 7 shows current against time for an alternative method of operating the apparatus of FIG. 1;

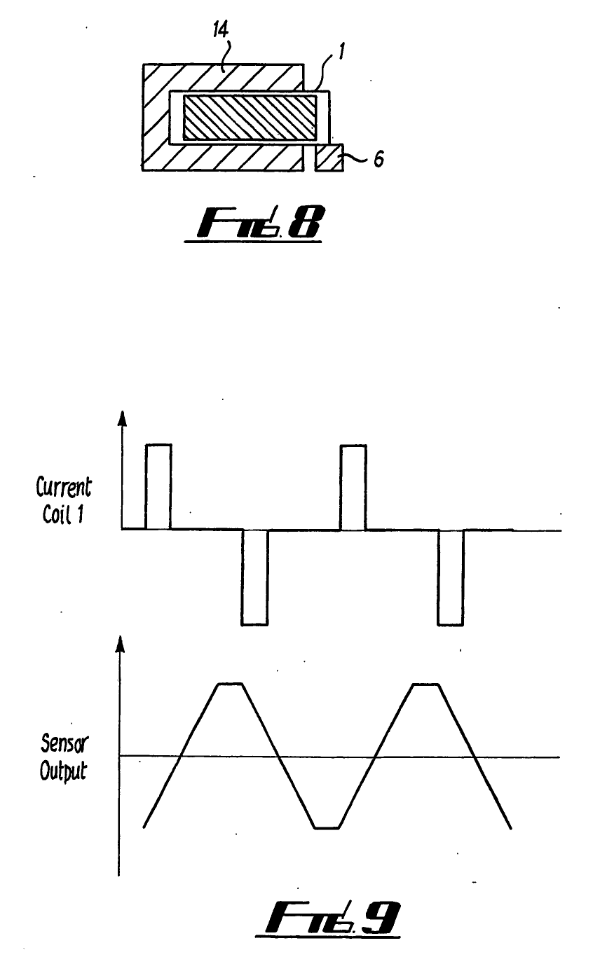

FIG. 8 is a schematic diagram of alternative apparatus embodying the invention; and

FIG. 9 shows drive current together with sensor output for the apparatus of FIG. 8;

The apparatus of FIG. 1 comprises a measurement unit and a separate sample chamber 1 which, in use, is inserted into the measurement unit.

In this embodiment, the sample chamber 1 is defined within a laminated slide-like structure (not shown) hereinafter referred to as a strip. The material of the structure which defines the chamber 1 is non-magnetic. The chamber 1 is substantially rectangular in shape and has a length of about 4 mm and a width of about 1.2 mm internally. The chamber 1 contains a rare earth magnetic body of substantially cuboid shape. The width of the body is about 200 microns less than that of the chamber, its height is about 200 microns less than that of the chamber, and its length is about 1.5 mm less than that of the chamber. The body 2 is magnetised along its rotational (long) axis. The chamber 1 also contains a dry reagent 3 for blood clotting distributed about the internal surface of the chamber 1. A suitable clotting agent is recombinant human tissue factor (Innovin®).

A capillary extends from a point on the strip remote from the chamber 1 into the chamber 1. In use, a sample of blood placed at sample-receiving opening of the capillary flows along the capillary, under capillary action, into the chamber 1. The magnetic body 2 fills over half the volume of the chamber and enhances filling of the chamber by capillary action.

The measurement unit comprises first and second spaced apart substantially coaxial solenoids 4,5. A Hall effect sensor 6 is disposed between the solenoids 4,5, displaced from the common axis of the solenoids so that it lies adjacent the sample chamber 1 when the sample chamber is introduced coaxially between the solenoids. The measurement unit 1 also includes various associated electrical circuitry (see especially FIG. 2) including a microprocessor 7. The unit also comprises a power supply (not shown), display 8, a resistive heating element 9 for heating a sample to be analysed and amplifiers 10 respectively associated with the sensor 6 and each solenoid 4,5.

The measurement unit has a support, not shown, for the strip so that when a strip is engaged by the support the chamber 1 lies between and substantially on the axis of the solenoids 4, 5. In this disposition the Hall Effect magnetic field sensor 6 lies in close proximity to the chamber.

The resistive heating element 9 is also located so that it is associated with the chamber 1 when the strip is inserted into the measurement unit 1, so that it is operative to heat a sample in the chamber 1 to a temperature of 37° C. In an alternative embodiment no resistive heating element is provided. Instead, any necessary heating of a sample in the chamber 1 is achieved by driving one or both solenoids 4,5 with a high frequency alternating current to generate an alternating magnetic field and cause inductive heating of the body 2 in the chamber 1 and thereby heat any sample in the chamber 1.

The microprocessor 10 is operative, amongst other things, to control supply of current to the two solenoids 4,5 by means of their respective amplifiers 10.

The Hall Effect sensor 6 is connected to its amplifier 10 which supplies the output of the sensor to the microprocessor via ADC circuitry (not shown).

In use a user switches on the measurement unit and inserts a strip containing a sample chamber 1 so that the chamber 1 is positioned between the solenoids 4,5.

If necessary, the microprocessor 8 causes the chamber 1 to be heated to a temperature of about 37° C. The microprocessor is able to determine the change in output of the Hall effect sensor 6 and thereby measure the temperature of the chamber 1. Other techniques are of course possible, including measurement of the resistance of the heating element 9, or provision of a separate thermal sensor.

In other embodiments, heating of the chamber to 37° C. is not necessary. Other e.g. lower temperatures can be used since with knowledge of the temperature of the sample the times determined by apparatus of the invention can be corrected to the values that would be achieved if the standard 37° C. were used. In one embodiment, no heating is used, and the temperature measured, e.g. by a sensor, and the necessary corrections applied.

When the chamber 1 is at the desired temperature a user is prompted to place a blood sample into the chamber 1. At the same time the solenoids 4,5 are alternately energised to cause the body 2 to vibrate within the chamber 1 at a frequency of about 5 to 10 Hz. This serves to enable rapid filling of the chamber 1 and mixes blood flowing into the chamber with the reagent 3. During this time the output of the Hall effect magnetic field sensor 6 is monitored. Due to the inherent magnetic susceptibility of blood the output from the sensor 6 rises rapidly as the chamber fills. When the output from the sensor 6 stabilises this indicates that the chamber has stopped filling.

The microprocessor 8 detects the stabilisation in output and in response thereto starts a timer to begin a measurement sequence. During this sequence the microprocessor alternately energises the solenoids 4,5 in a non-overlapping fashion so that they produce alternate magnetic fields of substantially opposite directions and records the output of the Hall effect sensor 6.

Referring to FIGS. 2 and 3 first one solenoid 4 is energised for a period of about 25 ms 11 allowing the magnetic field produced by the solenoid to form and settle. The magnetic component then travels for time 12 , the microprocessor 8 then waits for a similar period before measuring the output of the Hall effect sensor 6 during a measurement window 13 of duration about 100 ms which ends when the other solenoid 5 is energised with an opposite current, and the process is then repeated. When each solenoid is energised the resultant field causes the magnetic body 2 to experience a force urging it either towards or away from the energised solenoid, on solenoid causes a force to be exerted in one direction and the other solenoid in an opposite direction. When the chamber is filled with blood in a liquid state the applied magnetic fields cause the magnetic body to be shuttled from one end of the chamber 1 to the other causing the blood to flow from one end of the body 2 to the other through the annular space between the body 2 and the inside of the chamber 1. This causes turbulence in the blood in the chamber and keeps the blood and clotting reagent well mixed.

When neither solenoid is activated the output of the Hall effect sensor is dependent upon the position of the magnetic body within the chamber 1. FIG. 3 shows the output of the magnetic sensor 6 (disregarding the effect of the energised solenoids on the sensor output) as the magnetic body 2 moves to and fro within the chamber 1.

As the blood in the chamber 1 begins to clot the magnetic body 1 becomes bound within the forming clot matrix. The magnetic body will slow and eventually stop moving within the chamber 1. This has the effect of reducing, and then stopping variation in the output of the Hall effect sensor 6, as shown in FIG. 5.

The output of the sensor 6 is analysed by the microprocessor 8 to determine when movement of the magnetic body 2 ceases. This may be achieved using a variety of techniques. In one embodiment the output of the sensor is passed through a comparator which produces a square wave output every time the magnetic component moves, and a steady output whenever it stops. This may then be interpreted to give the clot point. In another embodiment the output value of the sensor 6 a predetermined time after energisation of one of the coils is recorded and compared with the equivalent value following subsequent energisation of the coil. Whilst the magnetic body is moved by the coils the monitored sensor output will remain substantially constant. When the body stops moving the output is likely to change.

When the magnetic body 2 stops moving this indicates that the sample has clotted. The timer is stopped and the time elapsed recorded is the coagulation time for the sample from which, with a knowledge of the clotting agent in the chamber 1, an INR value is calculated by the microprocessor 8 and operation of the solenoids ceased.

Where higher INRs are to be measured the microprocessor 8 is arranged to reduce the frequency of energisation of the solenoids 4,5 and/or the magnitude of the magnetic field they produce. This results in a reduction in power requirement by the apparatus whilst maintaining the accuracy of the measurement taken. The reduction in frequency and/or field strength where higher INR values are concerned reduces disturbance of clot formation by the magnetic body 2, important where the clot is potentially weaker. FIG. 6 shows sample time curves plotting the sample period (the period of movement of the magnetic body 2) against measured prothrombin times for measurement with 2 and 5% errors.

In addition to determining clotting time the apparatus can also measure the viscosity of a sample by measuring the time for the magnetic body 2 to travel across the known distance between opposite ends of the chamber 1.

FIG. 7 shows an alternative way of driving the solenoids 4,5. With this embodiment both solenoids are driven together and the current is reversed to cause the magnetic body to move to and fro within the chamber. Driving both solenoids together increases the magnetic field produced for a given drive current, reducing the power requirements of the apparatus as compared to the embodiment of FIG. 1.

FIG. 8 shows a further embodiment of the apparatus. Here the sample chamber 1 is associated with a single solenoid 14 and the Hall effect magnetic field sensor 6 is disposed adjacent one end of the chamber 1. The solenoid 14 extends around the chamber 1 so that the majority of the chamber 1 is disposed within the solenoid. In use the solenoid is driven with a reversing current, as shown in FIG. 9. Embodiments with a single solenoid can have a reduced power consumption and be more compact than embodiments with two solenoids.

In order to produce meaningful results the apparatus must be calibrated. The apparatus can be made self calibrating by providing more than one chamber disposed in the magnetic field produced by the solenoid or solenoids, each chamber with its own associated magnetic field sensor. With this arrangement each chamber contains a different amount and/or type of clotting agent. In use each chamber is filled at the same time with a sample of blood and the measurement sequence started. The relative times at which magnetic body in each chamber stops moving is then recorded. With a knowledge of the expected relative clotting times for each chamber, known because of the type and quantity of clotting reagent in each chamber it is possible to calibrate the apparatus.

The above embodiments are described by way of example only. Many variations are possible.

1-25. (canceled)

26. Apparatus for determining the coagulation status of a liquid, the apparatus comprising a chamber for holding a quantity of said liquid, a body disposed in the chamber and a magnetic device, the magnetic device co-operating with said chamber and being arranged in use to provide a magnetic field which causes the body to migrate to and fro within the chamber through uncoagulated liquid, wherein the body is other than a particle.

27. Apparatus as recited in claim 26 wherein means are provided to detect movement and/or position of the body within the chamber.

28. Apparatus as recited in claim 27 wherein the means to detect movement comprises a magnetic field sensor.

29. Apparatus as recited in claim 26 wherein the free volume within the chamber when the chamber contains the body is less than 10 μl.

30. Apparatus as recited in claim 26 wherein the chamber is formed in a disposable support strip which is removable from the apparatus.

31. Apparatus as recited in claim 26 wherein the chamber is elongate and of substantially uniform cross-section.

32. Apparatus as recited in claim 31 wherein the chamber is between 3 and 5 mm in length.

33. Apparatus as recited in claim 26 wherein the body is elongate and has a cross-section of substantially the same shape as the cross-section of the chamber.

34. Apparatus as recited in claim 26 wherein the body is dimensioned in cross-section so that there is a clearance of at least 50 microns between the body and walls of the chamber.

35. Apparatus as recited in claim 34 wherein the clearance is less than 300 microns.

36. Apparatus as recited in claim 26 wherein the length of the chamber and body may be chosen so that the body can move at least 0.5 mm to and fro within the chamber.

37. Apparatus as recited in claim 26 wherein the body can move a maximum of 2 mm to and fro within the chamber.

38. Apparatus as recited in claim 26 wherein the body comprises a material which experiences a force when placed in a magnetic field.

39. Apparatus as recited in claim 26 wherein a clotting reagent is disposed in the chamber.

40. Apparatus for determining the coagulation status of a liquid, the apparatus comprising a chamber for holding a quantity of said liquid, a body disposed in the chamber and a magnetic device, the magnetic device co-operating with said chamber and being arranged in use to provide a magnetic field which causes the body to move to and fro within the chamber through uncoagulated liquid, wherein the cross-sectional area of the body measured in a plane generally perpendicular to its normal direction of travel in use is at least half that of the chamber in the same plane.

41. Apparatus as recited in claim 40 wherein means are provided to detect movement and/or position of the body within the chamber.

42. Apparatus as recited in claim 41 wherein the means to detect movement comprises a magnetic field sensor.

43. Apparatus as recited in claim 40 wherein the free volume within the chamber when the chamber contains the body is less than 10 μ.

44. Apparatus as recited in claim 40 wherein the chamber is formed in a disposable support strip which is removable from the apparatus.

45. Apparatus as recited in claim 40 wherein the chamber is elongate and of substantially uniform cross-section.

46. Apparatus as recited in claim 45 wherein the chamber is between 3 and 5 mm in length.

47. Apparatus as recited in claim 40 wherein the body is elongate and has a cross-section of substantially the same shape as the cross-section of the chamber.

48. Apparatus as recited in claim 40 wherein the body is dimensioned in cross-section so that there is a clearance of at least 50 microns between the body and walls of the chamber.

49. Apparatus as recited in claim 48 wherein the clearance is less than 300 microns.

50. Apparatus as recited in claim 40 wherein the length of the chamber and body may be chosen so that the body can move at least 0.5 mm to and fro within the chamber.

51. Apparatus as recited in claim 40 wherein the body can move a maximum of 2 mm to and fro within the chamber.

52. Apparatus as recited in claim 40 wherein the body comprises a material which experiences a force when placed in a magnetic field.

53. Apparatus as recited in claim 40 wherein a clotting reagent is disposed in the chamber.

54. A method of determining the coagulation status of a liquid sample comprising the steps of: providing a sample of liquid in a chamber containing a body and applying a magnetic field to the chamber to cause the body to move to and fro within the chamber through uncoagulated liquid, wherein the body is other than a particle.

55. A method as recited in claim 54 comprising the steps of cyclically providing a first and a second magnetic field, said first magnetic field causing the body to move in a first direction and said second magnetic field causing the body to move in a second direction, the second direction being opposite to the first.

56. A method as recited in claim 55 wherein each field is provided as a short pulse, with a field free period between the short pulses.

57. A method as recited in claim 56 wherein the duration of each pulse is less than 500 ms.

58. A method as recited in claim 54 wherein the body is caused to move to and fro within the chamber at a frequency of between 0.1 and 10 Hz.

59. A method as recited in claim 54 wherein the magnitude of the magnetic field is less than 25 mT.

60. A method as recited in claim 54 further comprising the step of detecting movement and/or position of the body using a magnetic field sensor.

61. A method as recited in claim 54 wherein a clotting reagent is disposed in the chamber prior to introduction of a sample to be analysed.

62. A method of determining the coagulation status of a liquid disposed in a chamber, comprising the step of using at least one magnetic field to detect the movement and/or position of a body within said liquid, wherein the body comprises a material which experiences a force when placed in said at least one magnetic field, and further wherein said body is other than a particle.

63. A method of determining the coagulation status of a liquid sample comprising the steps of: providing a sample of liquid in a chamber containing a body; applying a magnetic field to the chamber to cause the body to move to and fro within the chamber through uncoagulated liquid, wherein the cross-sectional area of the body measured in a plane generally perpendicular to its normal direction of travel in use is at least half that of the chamber in the same plane.

64. A method as recited in claim 63 comprising the steps of cyclically providing a first and a second magnetic field, said first magnetic field causing the body to move in a first direction and said second magnetic field causing the body to move in a second direction, the second direction being opposite to the first.

65. A method as recited in claim 64 wherein each field is provided as a short pulse, with a field free period between the short pulses.

66. A method as recited in claim 65 wherein the duration of each pulse is less than 500 ms.

67. A method as recited in claim 63 wherein the body is caused to move to and fro within the chamber at a frequency of between 0.1 and 10 Hz.

68. A method as recited in claim 63 wherein the magnitude of the magnetic field is less than 25 mT.

69. A method as recited in claim 63 comprising the step of detecting movement and/or position of the body using a magnetic field sensor.

70. A method as recited in claim 63 wherein a clotting reagent is disposed in the chamber prior to introduction of a sample to be analysed.
